(19) United States
(12) Patent Application Publication
(10) Pub. No.: US 2016/0106209 A1
(43) Pub. Date: Apr. 21, 2016

(54) CPAP MACHINE CABINET

(71) Applicant: Howard Miller Company, Zeeland, MI (US)

(72) Inventors: Howard Jack Miller, Holland, MI (US); Michael Gerard Wallace, Grand Rapids, MI (US)

(21) Appl. No.: 14/516,742

(22) Filed: Oct. 17, 2014

(51) Int. Cl. A47B 81/00; A47B 79/00; A47B 57/58; A61M 16/00; A47B 23/00

(52) U.S. Cl. CPC A47B 81/00; A61M 16/0003; A47B 23/00; A47B 57/585; A47B 79/00

(57) ABSTRACT

A cabinet for a CPAP machine includes a base assembly with two side panels, front and rear panels, a top panel slidably mounted atop the base assembly, and a pull-out tray mounted within the base assembly. The top panel is movable between a storage position in which the top panel encloses an upper end of the base assembly, and an access position in which the top panel allows top access into an interior of the base assembly. The pull-out tray is movable between a stowed position in which the pull-out tray is entirely within the base assembly and an extended position in which the pull-out tray extends at least partially out of the base assembly. Each of the side panels defines an opening at an upper end configured to enable a hose of the CPAP machine to pass from the interior of the base assembly to the exterior.

### BACKGROUND OF THE INVENTION

The present invention relates to a cabinet, and more particularly to a bedside cabinet for storing a CPAP machine.

Continuous positive airway pressure (CPAP) therapy is a common treatment for obstructive sleep apnea. Therapy typically involves a small machine that supplies a constant and steady air pressure, a hose, a mask or nose piece, and an electrical cord or battery pack. The CPAP machine is most often stored on the floor next to the bed, or on a nearby nightstand. Because of the associated electrical cord and hose, the CPAP machine can be quite unsightly and the machine components (e.g. the hose and electrical cord) can become tangled. Though most users find the CPAP therapy to be effective in resolving sleep apnea, one common complaint is related to how to store and organize the equipment and accessories that are required for the CPAP machine.

### SUMMARY OF THE INVENTION

The noted issues are addressed by the present cabinet, which is a piece of furniture designed to discreetly store a CPAP machine and its accessories. The cabinet includes a base assembly having two substantially vertical, spaced side panels, a front panel, and a rear panel. The cabinet also includes a top panel and a pull-out tray, each slidably mounted atop and within the base assembly, respectively.

In a disclosed embodiment, the top panel is movable between a closed position, in which the top panel encloses an upper end of the base assembly, and an access position, in which the top panel is at least partially translated forward to allow top access into an interior of the base assembly. The pull-out tray is adapted to support the CPAP machine and is movable between a stowed position in which the pull-out tray is disposed within the base assembly, and an extended position in which the pull-out tray at least partially extends from the base assembly.

Each of the side panels has a cutout or opening in an upper edge thereof. The openings enable a hose of the CPAP machine to pass from the interior of the base assembly to an exterior of the base assembly when the top panel is in the closed position.

These and other features and advantages of the invention will be more fully understood and appreciated by reference to the entire application including the specification, the claims, and the drawings.

### BRIEF DESCRIPTION OF THE DRAWINGS

FIG. 1 is a front perspective view of a cabinet according to one embodiment;

FIG. 2 is a front perspective view of the cabinet of FIG. 1, illustrating a front panel of the cabinet in an open position, and a pull-out tray of the cabinet in an extended position;

FIG. 3 is a front perspective view of the cabinet of FIG. 1, illustrating a top panel of the cabinet in an access position;

FIG. 4 is a cross-sectional view of the cabinet, taken along line IV-IV of FIG. 1;

FIG. 5 is a cross-sectional view of the cabinet, taken along line V-V of FIG. 1;

FIG. 6 is a front perspective view of the cabinet of FIG. 1, illustrating a CPAP machine supported on the pull-out tray;

FIG. 7 is a side perspective view of the cabinet of FIG. 1, illustrating the top panel moved to an access position; and

FIG. 8 is a side perspective view of the cabinet of FIG. 1, illustrating a hose and mask of the CPAP machine passing through an opening in a side panel of the cabinet.

### DESCRIPTION OF THE CURRENT EMBODIMENTS

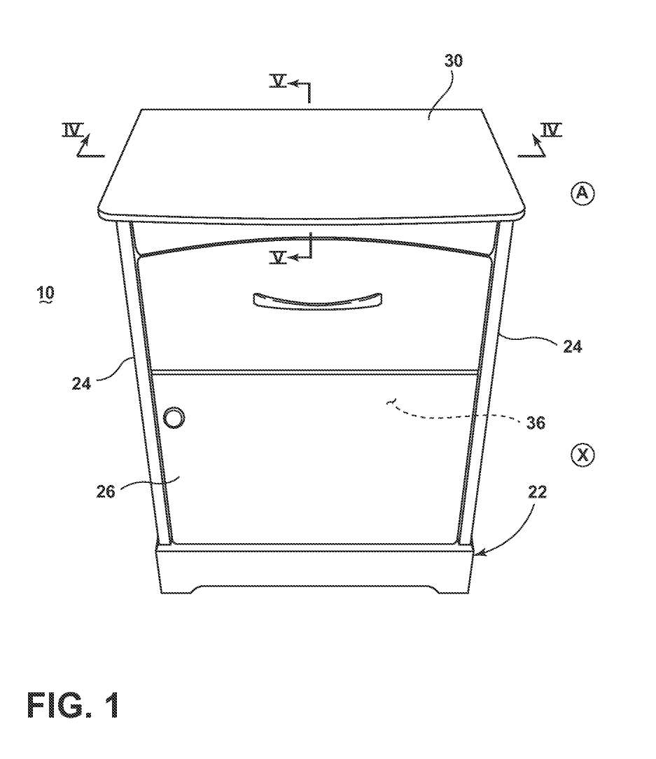

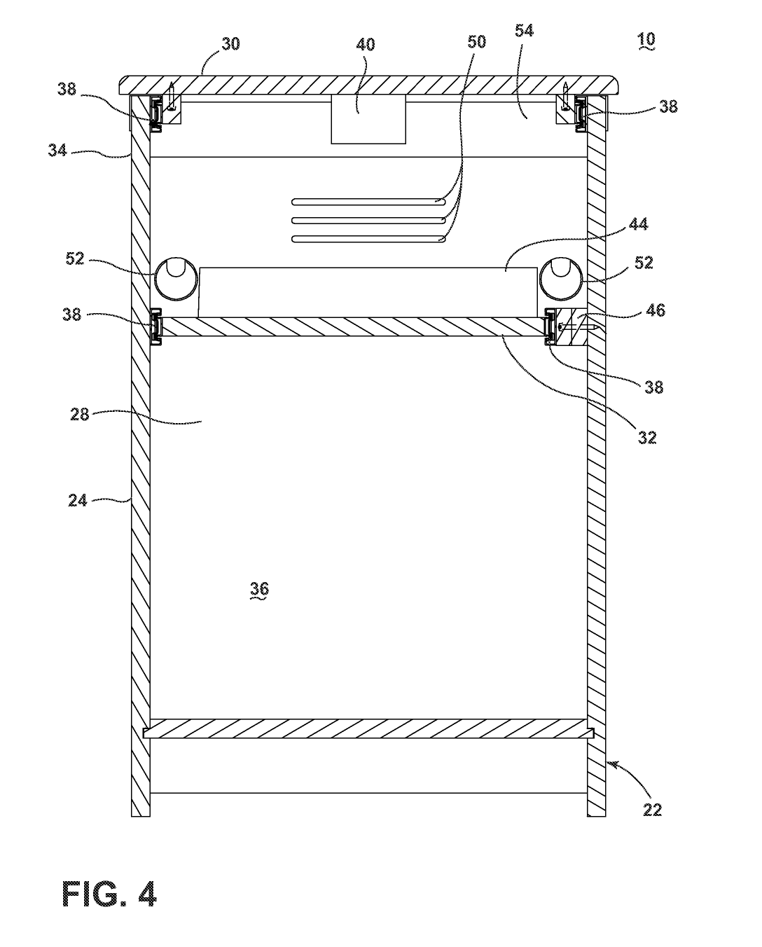

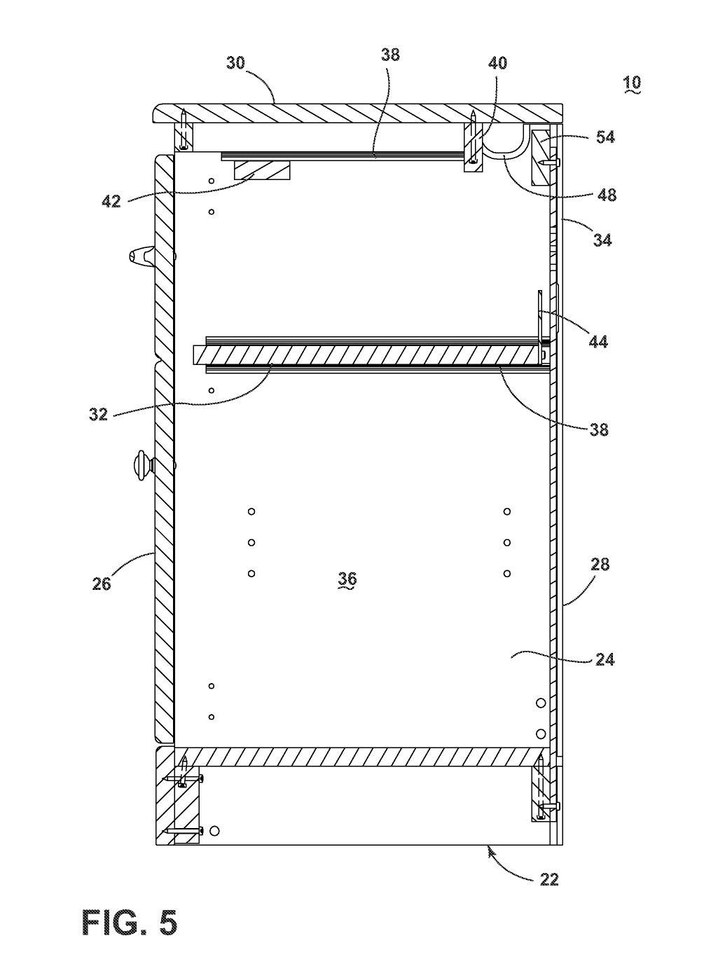

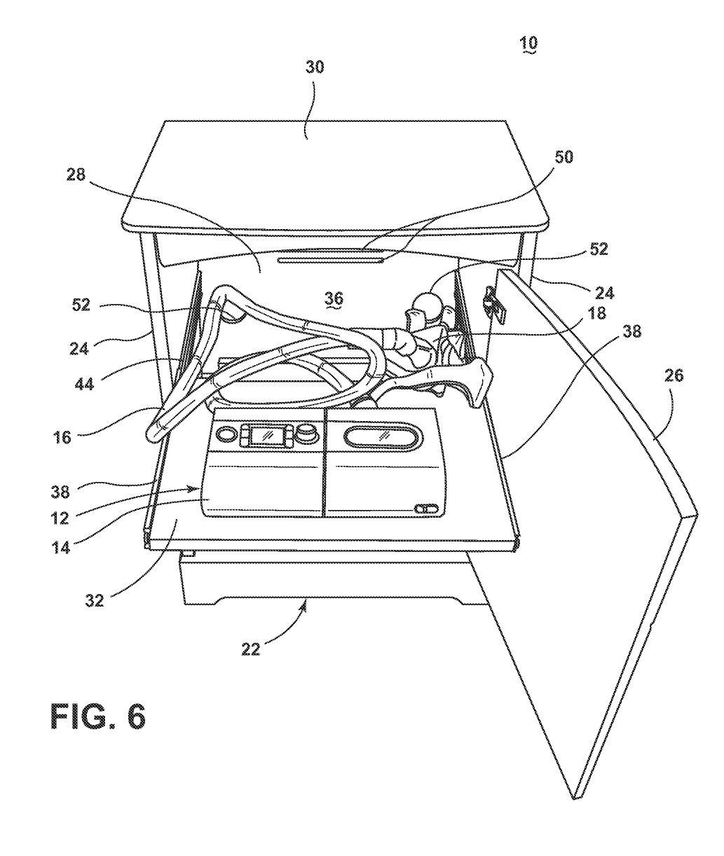

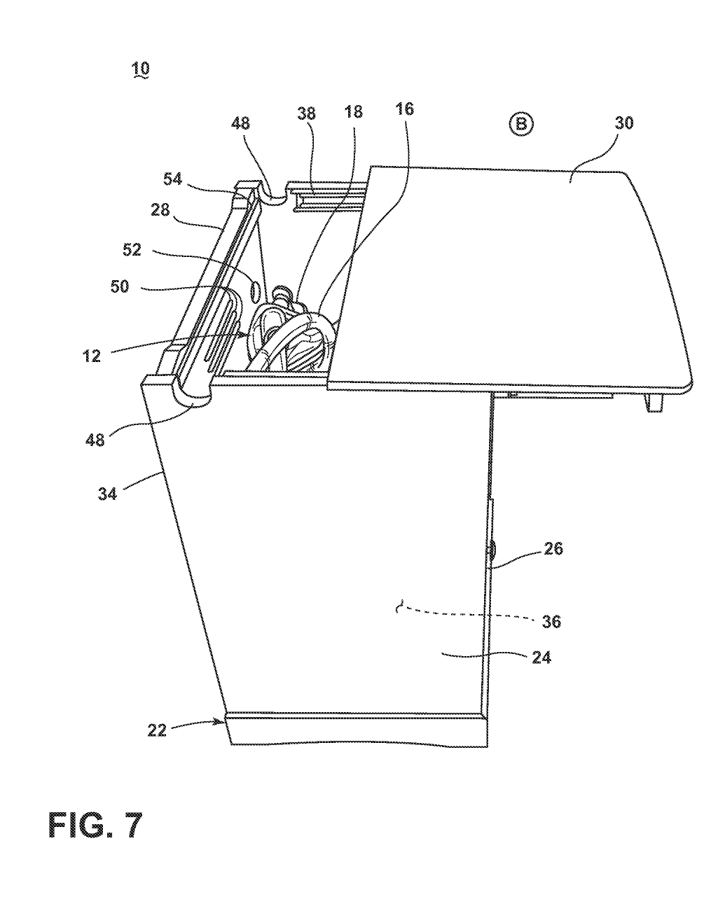

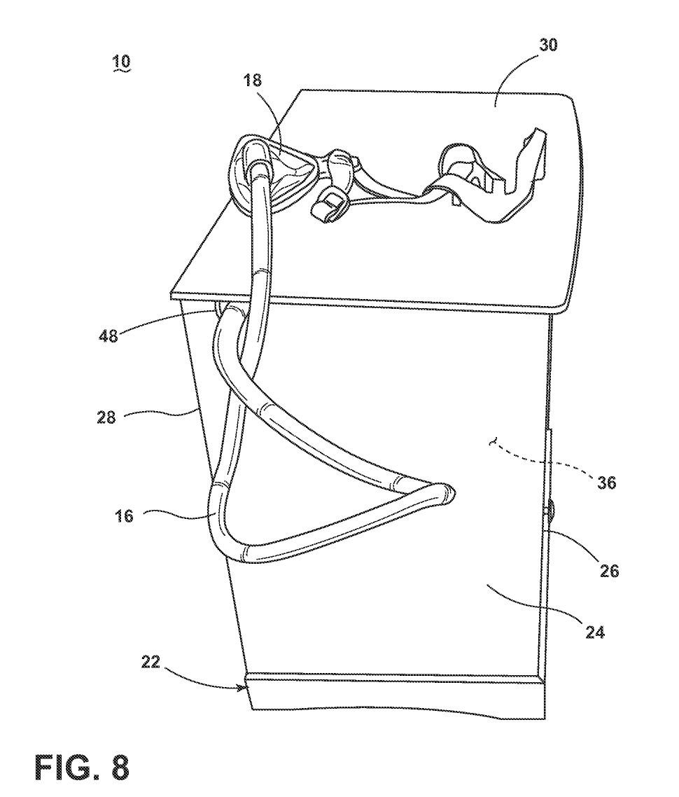

Referring to FIGS. 1-8, the numeral 10 generally designates a cabinet for the storage and use of a continuous positive airway pressure (CPAP) machine 12. CPAP machines are primarily used by patients for the treatment of sleep apnea. The CPAP machine is conventional and includes a flow generator 14 to provide airflow, a hose 16, an interface, such as a face mask 18, and an electrical cord (not shown). A humidifier may also be connected to the CPAP machine to add moisture to the low humidity air. The cabinet 10 is a quality piece of furniture that may generally be used as a bedside table for discretely housing the CPAP machine when not in use, and for facilitating use of the CPAP machine during sleep. Further, the cabinet 10 may support items on its top surface and store items within the cabinet 10, as do conventional bedside tables or nightstands. For example, a bedside lamp, alarm clock, etc.

The cabinet 10 is a substantially rectangular cuboid member that includes a base assembly 22 having two substantially vertical, spaced side panels 24, a front panel 26, and a rear panel 28. The cabinet 10 also includes a top panel 30 that is slidably mounted atop the base assembly 22 and a pull-out tray 32 that is slidably mounted within the base assembly 22.

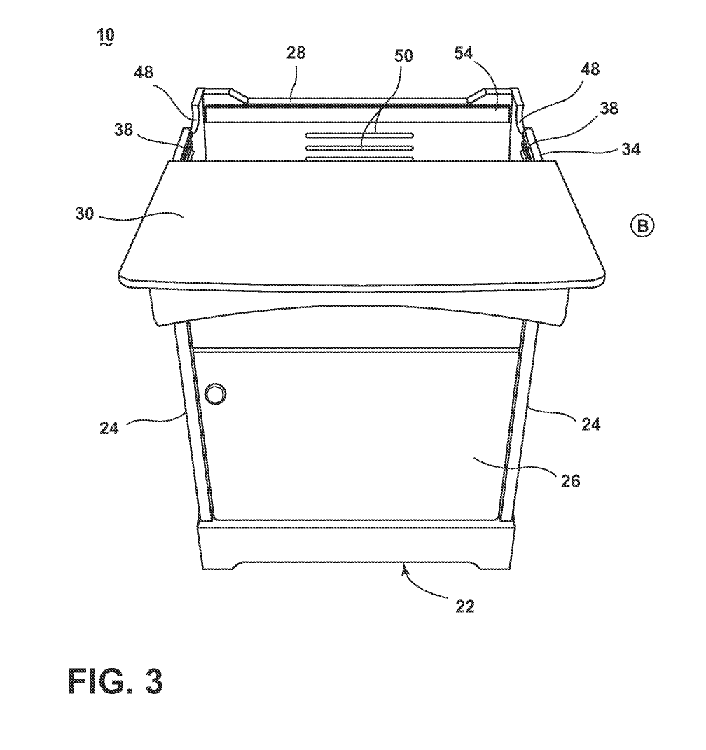

The top panel 30 is movable between a storage position A shown in FIG. 1, in which the top panel 30 encloses an upper end 34 of the base assembly 22, and an access position B shown in FIG. 3, in which the top panel 30 is at least partially translated forward to allow top access into an interior 36 of the base assembly 22. The top panel 30 is slidably affixed to the base assembly 22, and more specifically to the side panels 26, by horizontal slides 38, such as conventional drawer slides.

Additionally, the top panel 30 includes a stop block 40 that is mounted on an underside of the top panel 30. The stop block 40 is positioned a predetermined distance from the front edge of the top panel 30. A cooperative rail 42 extends horizontally between the spaced side panels 24. The rail 42 is also positioned a predetermined distance from the front edge of the top panel 30, such that when the top panel 30 is translated forward to the access position B, the stop block 40 contacts the rail 42, thereby preventing the top panel 30 from extending out further than desired from the base assembly 22.

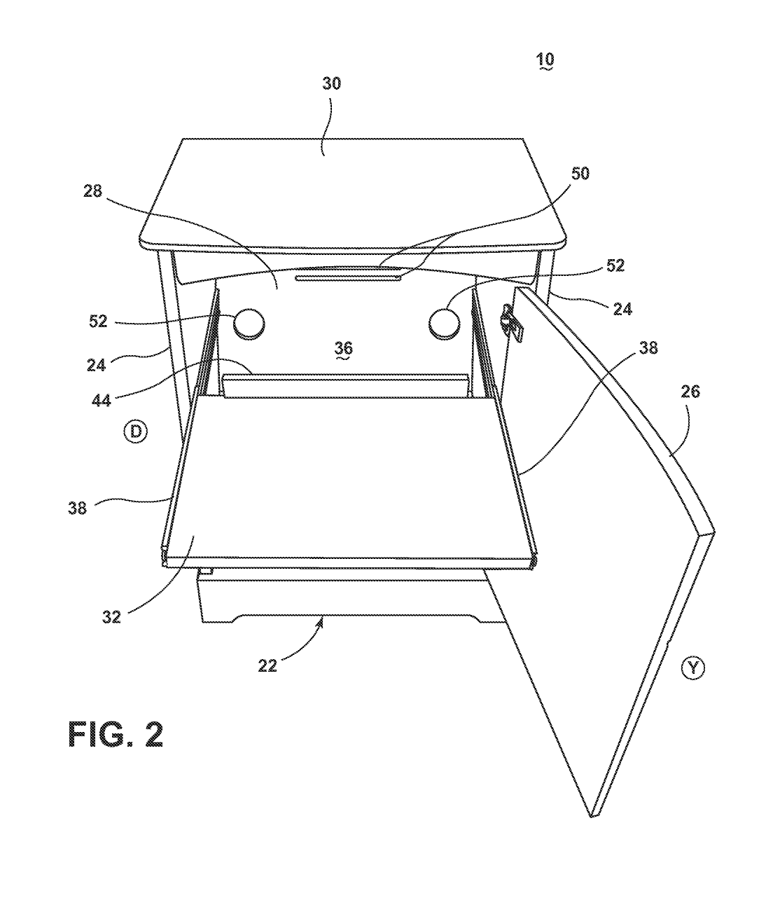

The front panel 26 is hingedly mounted to the base assembly 22 and pivots about a vertical axis between a closed position X shown in FIG. 1 and an open position Y shown in FIG. 2. The front panel 26 may include a knob or other hardware and functions as a simple vertically-hinged door.

The pull-out tray 32 is adapted to support the CPAP machine 12 and is movable between a stowed position C as in FIG. 1 in which the pull-out tray 32 is disposed within the base assembly 22 and an extended position D shown in FIG. 2 in which the pull-out tray 32 is at least partially translated forward out of the base assembly 22. The pull-out tray 32 includes a flange 44 disposed at a rear edge of the pull-out tray 32. The purpose of the flange 44 is to prevent items placed on the pull-out tray 32 from falling off the rear edge of the tray during movement of the pull-out tray 32 to the extended position D.

The pull-out tray 32 is mounted between the side panels 24 by first and second horizontal slides 38. Additionally, a spacer 46 is included between one of the first and second slides 38 and the adjacent side panel 24. More specifically, the spacer 46 is positioned on the same side of the base assembly 22 as which the front panel 26 is mounted. Including a spacer 46 on the stated side laterally shifts the pull-out tray 32 to ensure that the tray 32 is free from obstruction when the front panel 26 is in the open position Y and the pull-out tray 32 is translated to the extended position D. Optionally, a second spacer may be included on the opposite side.

The side panels 24 each include an opening 48 at an upper end thereof. The openings 48 are substantially U-shaped members accessible from the top edge of the side panels 24. The openings 48 are configured to allow the hose 16 of the CPAP machine 12 to pass from the interior 36 of the base assembly 22 to an exterior of the base assembly 22. Accordingly, the openings 48 are appropriately sized to accommodate the hose 16. Furthermore, the cabinet has an opening 48 on each side so that the cabinet 10 can be on either side of the bed, depending on which side of the bed the user typically sleeps.

The rear panel 28 includes one or more ventilation slots 50 to provide proper ventilation and fresh air for the CPAP machine 12. Further, the rear panel 28 may include one or more access holes 52 through which the electrical cord of the CPAP machine 12 may pass from the interior 36 to the exterior of the base assembly 22. This enables the CPAP machine 12 to be plugged in to a nearby electrical outlet (not shown), while limiting visibility of the electrical cord from the front of the cabinet 10. Electrical cords of other objects contained within the cabinet 10 or on the top panel 30 may also pass through the access holes 52. Typically, electrical cords are routed to the opposite side of where the hose 16 is routed through opening 48 to avoid interference or entanglement of electrical cords with hose 16 and mask 18. Additionally, the rear panel 28 includes a top block rail 54 that extends horizontally between the spaced side panels 24. The top block rail 54 is positioned at a top end of the rear panel 28 such that a relief space is formed between the top block rail 54 and the top panel 30. The relief space allows electrical cords from items located on the top panel 30 to be routed into the cabinet 10 and back through the access holes 52.

The top panel 30 and pull-out tray 32 are independently translated. In other words, they do not move together unless moved at the same time. Further, the area under the pull-out tray 32 can be utilized to house a variety of other items, including an associated humidifier, a bottle of distilled water, CPAP cleaning products and wipes, etc.

The cabinet 10 and its components can be made of any suitable material, particularly of finished or unfinished wood, wood composite, laminates, melamine, or vinyl.

### Operation

The cabinet 10 facilitates easy storage and use of a CPAP machine 12. The CPAP machine 12, including the associated hose 16 and mask 18, can be placed on the pull-out tray 32 and stored within the base assembly 22. The CPAP machine's electrical cord can be pushed through one of the access holes 52 in the rear panel 28 and plugged into a nearby electrical outlet. With the top panel 30, pull-out tray 32, and front panel 26 all in their respective storage A, stowed C, and closed X positions, the cabinet 10 appears to be a conventional bedside table or night stand. The CPAP machine 12, hose 16, and mask 18 are all stored inside the cabinet 10 and out of sight. Additionally, a number of common bedside items can be placed on the surface of the top panel 30; for example, a bedside lamp, alarm clock, reading material, etc.

To use the CPAP machine 12, a user can slide/translate the top panel 30 forward, from the storage position A to the access position B shown in FIG. 3. The access position B allows access from above into the interior 36 of the base assembly 22, and specifically allows the user access to the CPAP machine 12 placed on the pull-out tray 32. With the top panel 30 in the access position B, the user can pull the hose 16 and mask 18 out of the base assembly 22, and place the hose 16 (from above) into the opening 48 in the side panel 24 preferably adjacent the bed. Once the hose 16 is in position, the top panel 30 can be slid back to the storage position A, again concealing the CPAP machine 12 within the cabinet 10.

The cabinet 10 allows easy access to and use of a CPAP machine 12, while at the same time providing a neat and aesthetically pleasing storage solution for users who keep a CPAP machine 12 near their bed. Additionally, instead of having to take the entire CPAP machine 12, hose 16, and mask 18 in and out of the cabinet every morning and evening, the user is able to leave the equipment in the cabinet 12 and simply slide the top panel 30 open and closed when the hose 16 and mask 18 are needed. Another benefit of the present cabinet 10 is that the user can access the interior 36 of the cabinet 12 without disturbing the items placed on the top panel 30.

The above descriptions are those of the current embodiments of the invention. Various alterations and changes can be made without departing from the spirit and broader aspects of the invention as defined in the appended claims, which are to be interpreted in accordance with the principles of patent law including the doctrine of equivalents. Any reference to elements in the singular, for example, using the articles “a,” “an,” “the,” or “said,” is not to be construed as limiting the element to the singular.

1. A CPAP machine cabinet comprising:
a base assembly having two substantially vertical, spaced side panels, a front panel extending between the side panels, and a rear panel extending between the side panels, each of the side panels including a top edge, each of the side panels defining an opening that opens through the top edge of the side panel, each opening configured to allow a hose of a CPAP machine to be placed into the opening from above and thereby to pass from an interior of the base assembly to an exterior of the base assembly;
a top panel slidably mounted atop the base assembly and movable between a storage position, in which the top panel encloses an upper end of the base assembly, and an access position, in which the top panel is at least partially translated forward to allow top access into the interior of the base assembly, the openings in the side panels being covered by the top panel when in the storage position and being uncovered by the top panel in the access position; and
a pull-out tray slidably mounted within the base assembly and adapted to support the CPAP machine and is movable between a stowed position in which the pull-out tray is disposed within the base assembly, to an extended position in which the pull-out tray at least partially extends forward of the base assembly.

2. The CPAP machine cabinet of claim 1 wherein the rear panel defines ventilation slots.

3. The CPAP machine cabinet of claim 1 wherein the front panel comprises a door hingedly mounted to the base assembly.

4. The CPAP machine cabinet of claim 1 further including first and second horizontal slides mounted to the pull-out tray and a spacer joined to one of the first and second horizontal slides, such that the pull-out tray is free from obstruction when the front panel is in an open position and the pull-out tray is translated to the extended position.

5. The CPAP machine cabinet of claim 1 wherein the pull-out tray includes a flange disposed at a rear edge thereof.

6. The CPAP machine cabinet of claim 1 wherein the top panel and pull-out tray are independently translated.

7. The CPAP machine cabinet of claim 1 further including a stop block disposed on an underside of the top panel and a rail that extends horizontally between the spaced side panels, such that when the top panel is translated forward to the access position the stop block contacts the rail, thereby preventing the top panel from extending out further than desired from the base assembly.

8.-15. (canceled)

16. A CPAP machine cabinet comprising:
a base assembly having two substantially vertical side panels, a front panel extending between the side panels, and a rear panel extending between the side panels;
a top panel slidably mounted atop the base assembly, the top moveable between a storage position, in which the top panel encloses a top portion of the base assembly, and an access position, in which the top panel is at least partially translated forward to allow top access into an interior of the base assembly; and
a pull-out tray slidably mounted within the base assembly, the pull-out tray adapted to support a CPAP machine and movable between a stowed position in which the pull-out tray is within the base assembly and an extended position in which the pull-out tray is at least partially forward of the base assembly,
wherein each of the side panels includes a top edge, each of the side panels defining an opening that opens through the top edge of the side panel, each opening configured to allow a hose of the CPAP machine to be placed into the opening from above and thereby to pass from the interior of the base assembly to an exterior of the base assembly, and
wherein the openings in the side panels are covered by the top panel when in the storage position and are uncovered when the top panel is in the access position.

17. The CPAP machine cabinet of claim 16 wherein the front panel is hingedly mounted to the base assembly, and may be pivoted about a vertical axis between closed and open positions.

18. The CPAP machine cabinet of claim 16 wherein the pull-out tray includes a flange disposed at a rear edge thereof.

19. The CPAP machine cabinet of claim 16 further including a stop block disposed on an underside of the top panel and a rail that extends horizontally between the spaced side panels, such that when the top panel is translated forward to the access position the stop block contacts the rail, thereby preventing the top panel from extending out further than desired from the base assembly.

20. The CPAP machine cabinet of claim 19 further including first and second horizontal slides mounted to the pull-out tray and a spacer joined to one of the first and second horizontal slides, such that the pull-out tray is free from obstruction when the front panel is in an open position and the pull-out tray is translated to the extended position.
